(19) United States
(12) Patent Application Publication
(10) Pub. No.: US 2017/0128116 A1
(43) Pub. Date: May 11, 2017

(54) TORQUE LIMITING DEVICES AND METHODS OF USE

(71) Applicant: GLOBUS MEDICAL, INC., AUDUBON, PA (US)

(72) Inventors: Noah Hansell, King of Prussia, PA (US); Brad Juchno, Yardley, PA (US)

(21) Appl. No.: 14/934,917

(22) Filed: Nov. 6, 2015

(51) Int. Cl. A61B 17/88; B25B 23/142; B25B 15/04; B25B 23/14

(52) U.S. Cl. CPC A61B 17/8875; B25B 23/141; B25B 23/1427; B25B 15/04

(57) ABSTRACT

Exemplary embodiments of apparatuses and methods of a torque limiting device are provided. In one embodiment, a driving device comprises a torque limiting clutch including a lower ratchet, the lower ratchet including a plurality of ramps, an upper ratchet, and an upper ratchet including a plurality of bearings. The driving device further comprises a compression spring, wherein the compression spring is configured to exert a force on the upper ratchet so that the plurality of bearings interface with the plurality of ramps, wherein the plurality of bearings ride on the plurality of ramps when a torque is created by a rotation of the driving device thereby limiting an amount of torque.

### FIELD OF THE DISCLOSURE

The present disclosure relates to exemplary embodiments of apparatuses and methods for a torque limiting device.

### BACKGROUND INFORMATION

During various surgical procedures it is necessary to insert bone screws to hold bones, bone plates and other surgical implants in position. Typically, these procedures require the bone screw to be loaded onto a screwdriver and then passed to the surgeon for insertion. Upon insertion, the screwdriver is turned multiple times until the surgeon achieves sufficient depth and holding power of the bone screw for the particular procedure.

At times, it is difficult for the surgeon to gauge the torque needed for each of these insertions. If the wrong of amount of torque is applied, it may cause the screw to strip once it meets a certain resistance or it may cause unacceptable levels of stress and strain upon the surgical repair site resulting to damage of the repair site. The damage to the screw or the repair site can make it nearly impossible to properly secure a surgical implant. Accordingly, a system and method are needed to address the shortfalls of conventional techniques and to provide other new and innovative features.

### SUMMARY OF EXEMPLARY EMBODIMENTS OF THE PRESENT DISCLOSURE

At least some of the above described problems can be addressed by exemplary embodiments of the apparatuses and methods according to the present disclosure. For example, using such exemplary embodiments, it is possible to provide an anti-torque driver that limits an amount of torque applied to an object.

In some exemplary embodiments, a driving device can comprise a torque limiting clutch including: a lower ratchet including a plurality of ramps, an upper ratchet including a plurality of bearings, and a compression spring, the compression spring configured to exert a force on the upper ratchet so that the plurality of bearings interface with the plurality of ramps, wherein the plurality of bearings ride on the plurality of ramps when a torque is created by a rotation of the driving device thereby limiting an amount of torque.

In some exemplary embodiments, as the plurality of bearings ride up the plurality of ramps, the upper ratchet is pushed toward the compression spring thereby applying a compression force that maxes out when the plurality of bearings reach a top of the ramps thereby limiting the amount of torque.

In some exemplary embodiments, the driving device further includes a shaft, wherein the torque limiting clutch controls the amount of torque being applied to a surgical fixation element attached to the shaft.

In some exemplary embodiments, the plurality of ramps can be steeper in a clockwise direction than a counter-clockwise direction, steeper in a counter-clockwise direction than a clockwise direction or have equal steepness in both a counter-clockwise direction and a clockwise direction.

In some exemplary embodiments, the torque limiting clutch for a driving device can further comprise a retaining ring for retaining the plurality of bearings to the upper ratchet. In some exemplary embodiments, the torque limiting clutch for a driving device can further comprise: a thrust washer, the trust washer transmitting and resolving axial forces in the driving device thereby keeping the torque-limiting clutch aligned along the shaft. In some exemplary embodiments, the torque limiting clutch for a driving device can further comprise: a compression nut applying a force to the compression spring so that an appropriate amount of force is exerted onto the upper ratchet with respect to the lower ratchet. In some exemplary embodiments, the appropriate amount of force can be approximately 0.5 to approximately 3 pounds of pressure.

In some exemplary embodiments, the driving device can be a screwdriver or a T-Handle screwdriver geometrically suited to exert a higher torque load to an object. In some exemplary embodiments, the lower ratchet includes backlashes that prevent the torque-limiting clutch from moving opposite its direction of rotation.

In some exemplary embodiments, a driving device can be provided, comprising a shaft, the shaft including an upper section having a proximal end and a distal end and a lower section having a proximal end and a distal end, a handle, the handle being attached to the proximal end of the upper section, a torque limiting clutch, the torque limiting clutch including a lower ratchet having a plurality of discs and backlashes and an upper ratchet having bearing assembly, the lower ratchet being attached to the proximal end of the lower section and the upper ratchet being attached to the distal end of the upper section, and a compression spring, the compression spring being placed on the upper section of the shaft between the upper ratchet and the handle, the compression spring exerting a force on the upper ratchet so that the bearing assembly interfaces with the plurality of discs and backlashes.

In some exemplary embodiments, the bearing assembly rides on the plurality of discs when a torque is created by a rotation of the driving device. In some exemplary embodiments, as the bearing assembly rides up the plurality of discs, the upper ratchet is pushed toward the compression spring thereby applying a compression force that maxes out when the bearing assembly reaches a top of the plurality of discs thereby limiting an amount of torque. The driving device can be a T-Handle screwdriver geometrically suited to exert a higher torque load to an object. The backlashes can prevent the torque limiting clutch from moving opposite its direction of rotation.

These and other objects, features and advantages of the present disclosure will become apparent upon reading the following detailed description of embodiments of the present disclosure, when taken in conjunction with the appended claims. It should be understood that the detailed description and specific examples, while indicating the preferred or exemplary embodiments of the disclosure, are intended for purposes of illustration only and are not intended to limit the scope of the disclosure.

### BRIEF DESCRIPTION OF THE DRAWINGS

The foregoing and other exemplary objects of the present disclosure will be apparent upon consideration of the following detailed description, taken in conjunction with the accompanying exemplary drawings and claims, in which like reference characters refer to like parts throughout, and in which:

FIG. 1 illustrates a side perspective view of a torque limiting device according to an exemplary embodiment of the present disclosure;

FIG. 2 illustrates a side perspective view of a torque limiting device according to another exemplary embodiment of the present disclosure;

FIG. 3 illustrates a side perspective view of an upper portion of a torque limiting device according to an exemplary embodiment of the present disclosure;

FIG. 4 illustrates a close-up perspective view of a clutch of a torque limiting device according to an exemplary embodiment of the present disclosure;

FIG. 5 illustrates a side view of a torque limiting device according to an exemplary embodiment of the present disclosure;

FIG. 6 illustrates a side perspective view of a torque limiting device according to another exemplary embodiment of the present disclosure;

FIG. 7 illustrates a side perspective view of a torque limiting device according to an exemplary embodiment of the present disclosure;

FIG. 8 illustrates a side view of a clutch of a torque limiting device according to an exemplary embodiment of the present disclosure; and

FIG. 9 a side perspective view of a clutch of a torque limiting device according to an exemplary embodiment of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION OF EXEMPLARY EMBODIMENTS OF DISCLOSURE

Exemplary embodiments of the apparatuses and methods of the present disclosure will now be described with reference to the figures. The following description of the various embodiments is merely exemplary in nature and is in no way intended to limit the scope of the disclosure, its application, or uses.

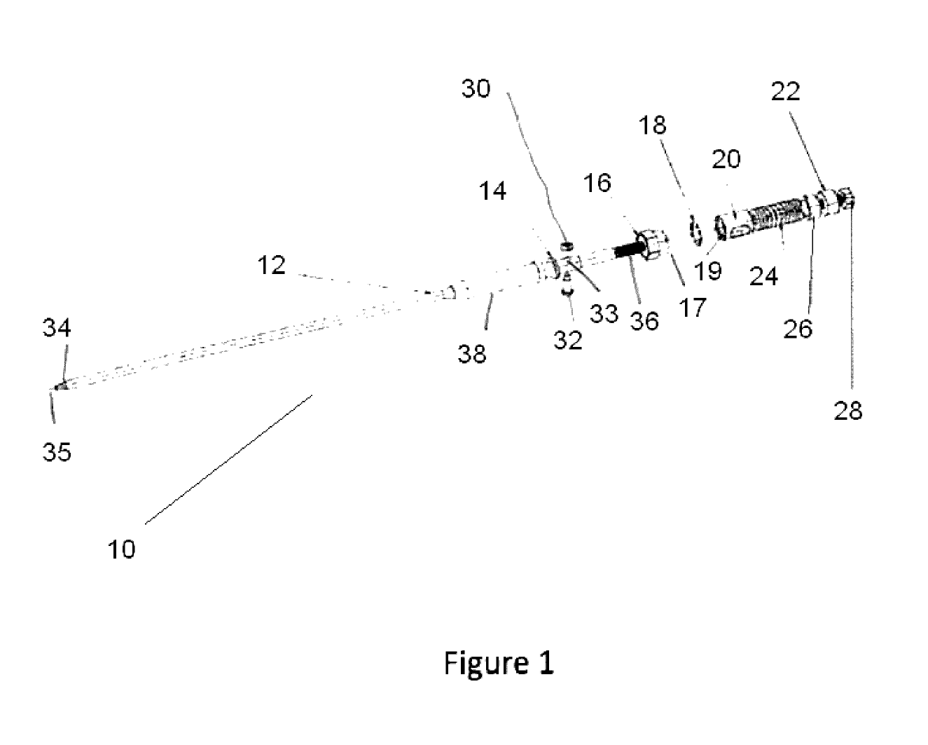

As shown in FIG. 1, a driving device 10 comprises a shaft 12. In some exemplary embodiments, the shaft 12 can be an elongated stem having a tip 34 configured to couple with an object 35, e.g., a head of a surgical fixation element, such as a screw. On an opposite end, the shaft 12 can comprise screw threads 36 for receiving a retaining nut 28 and a compression nut 26. The mid-section of the shaft 12 can include a hub 38 that allows for securement of a handle (not shown) to the shaft 12. The shaft 12 can also include a through hole 33 for receiving a lower ratchet retaining screw 32. In one embodiment, the through hole 33 can be located slightly above the hub 38.

On the upper portion of the shaft 12, between the hub 38 and the screw threads 36, a torque-limiting clutch can be positioned. The torque-limiting clutch can advantageously be used to control an amount of torque being applied to the surgical fixation element 35 via the shaft 12, thereby reducing the risk of damage to the surgical fixation element 35. In some embodiments, the torque-limiting clutch can include a thrust washer 14, a lower ratchet 16, a retaining ring 18, an upper ratchet 20, a compression spring 24, a spacer 26, a compression nut 26, a retaining nut 28, a lower ratchet retaining screw 32 and a lower ratchet retaining nut 30.

In some embodiments, the lower ratchet 16 can include ramps 17. The ramps 17 can be positioned on a top face thereof. That is, the lower ratchet 16 can include a series of ramped serrations that are located at the edge of the upper face. In some embodiments, and the ramps 17 surround the circumference of the upper face. In some embodiments, the ramps can be steeper in the clockwise direction than counter-clockwise direction, which limits torque in the opposite direction of the use. In other implementations, the ramps can be steeper in the counter-clockwise or the ramps can have the same steepness in either direction. The angle of the ramps can range, e.g., from approximately 20 to approximately 45 degrees, which can depend on the application of use and the torque needed for the application.

The thrust washer 14 can be placed between the hub 38 and a bottom face of the lower ratchet 16. The thrust washer can be a long-wearing flat bearing in the shape of a washer that transmits and resolves axial forces in the driving device 10 thereby keeping the torque-limiting clutch aligned along the shaft 12.

The upper ratchet 20 can include a bearing system 19. In some embodiments, the bearing system 19 comprises a number of ball bearings or other type of ball assembly, located on a bottom face thereof. The bearing system 19 can be held in place by the retaining ring 18 and can interface with the ramps 17 of the lower ratchet 16, as will be described more fully below. In some implementations, the top face of the upper ratchet 20 is in contact with a bottom portion of a compression spring 24 which is retained in position by a spacer 26, a compression nut 22 and a retaining nut 28.

During manufacture, the thrust washer 14, the lower ratchet 16, the retaining ring 18, the upper ratchet 20, the compression spring 24 and the spacer 26 can be slid onto the shaft. The lower ratchet can then be fixedly secured to the shaft by placing the lower ratchet retaining screw 32 into the through hole 33 and the lower ratchet retaining nut 30 secures the screw in place. The compression nut 26 is then screwed onto the threaded portion of the shaft and tightened until an appropriate amount of force is exerted onto the upper ratchet with respect to the lower ratchet. In some embodiments, approximately 0.25 to 5 pounds of pressure is exerted onto the upper ratchet. In other words, the driving device 10 can advantageously be calibrated based on the compression spring 24 to limit the amount of force that is applied to an object being driven by the torque-limiting clutch. Once the appropriate amount of force is achieved, the retaining nut 28 can be tightened against the compression nut 26 so that the compression nut 26 does not loosen during its use.

In use, as a handle is rotated by a user, the handle rotates the shaft so that a screw can be driven into an object. As the screw is driven deeper into the object, a torque is created. As the torque increases it allows the bearing system 19 of the upper ratchet 20 to ride on the ramps 17 of the lower ratchet 16. As the ball bearings 19 ride up the ramps, the upper ratchet 20 is pushed toward the compression spring 24. The compression spring 24, in turn, applies a compression force to the upper ratchet 20 that maxes out when the bearings reach a top of the ramps 17. This configuration serves to limit the amount of torque that can be applied to the object. Once the torque limit of the compression spring is achieved, the handle can be rotated but no further torque will be applied to the screw. In other words, if more torque is applied to the handle, the bearings will ride the ramps but no torque greater than the maximum torque can be delivered to the shaft tip 34.

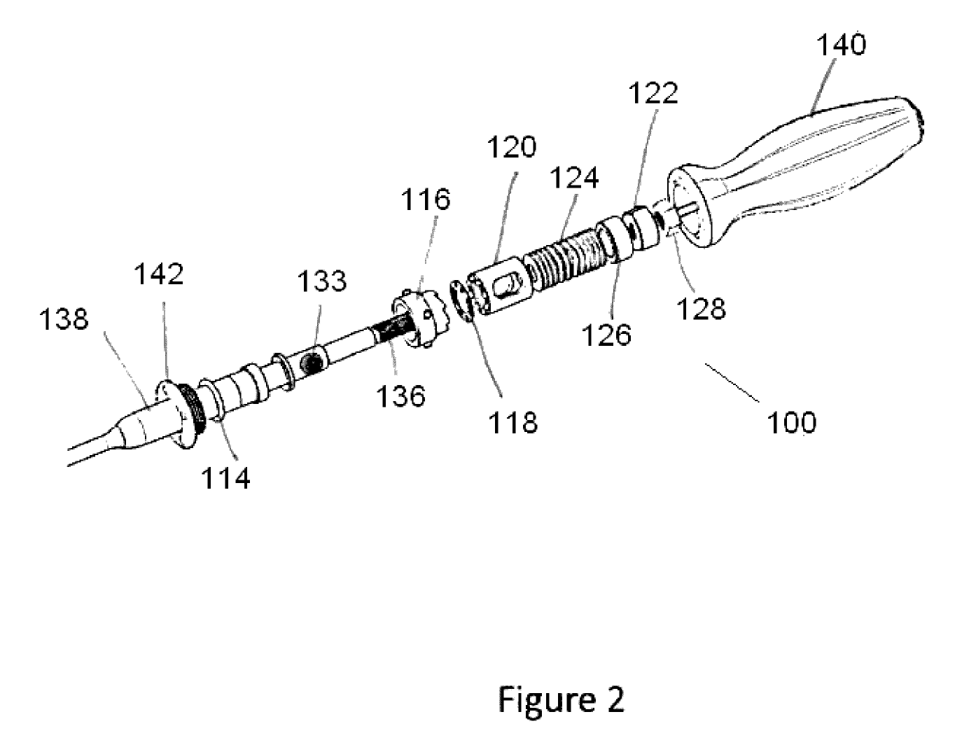

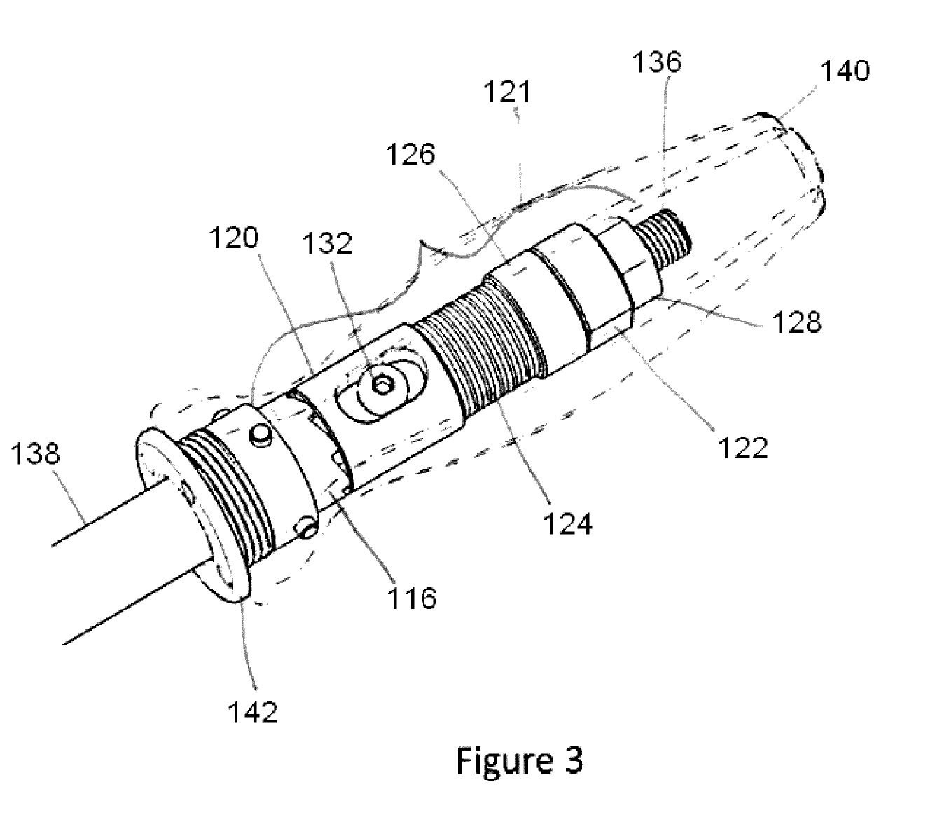

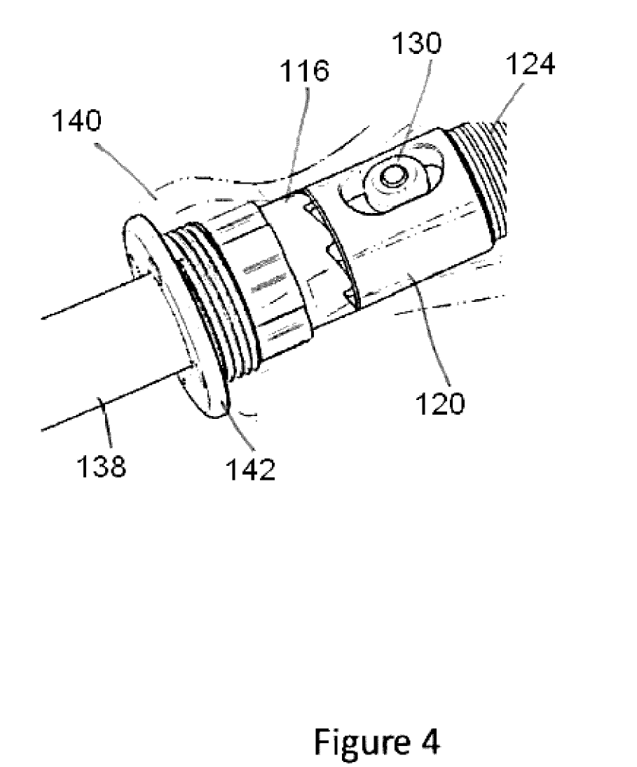

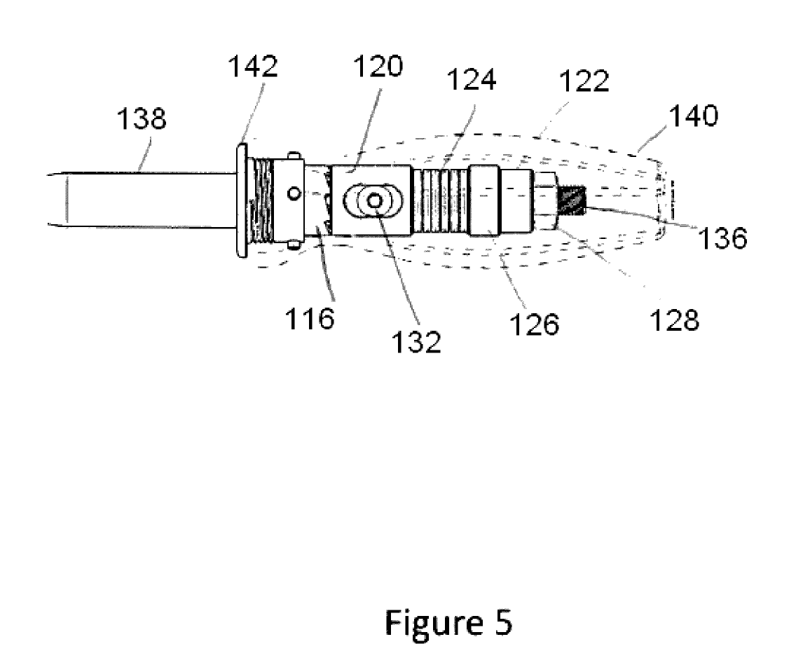

As shown in FIGS. 2-5, a torque limiting screwdriver 100 includes a shaft 112. The shaft 112 can be an elongated stem having a tip 34 (not shown) configured to couple with an object (not shown), e.g., a head of a surgical fixation element, at one end. At the other end, the shaft 112 can comprise screw threads 136 for receiving a retaining nut 128 and a compression nut 122 at the other end. The mid-section of the shaft 112 can include a hub 138 and handle retainer 142 that allows for securement of a handle 140 to the shaft 112. The shaft 112 can also include a through hole 133 for receiving a upper ratchet retaining screw 132. In one embodiment, the through hole 133 can be located above the hub 138.

On the upper portion of the shaft 112, between the hub 138 and the screw threads 136, a torque-limiting clutch 121 can be positioned. The torque-limiting clutch 121 can be used to control an amount of torque being applied to a surgical fixation element via the shaft 112. The torque-limiting clutch 121 can includes a thrust washer 114, a lower ratchet 116, a retaining ring 118, an upper ratchet 120, a compression spring 124, a spacer 126, a compression nut 126, a retaining nut 128, an upper ratchet retaining screw 132 and an upper ratchet retaining nut 130.

The lower ratchet 116 can include ramps 117 on a top face thereof. That is, the lower ratchet 116 includes a series of ramped serrations that are located at the edge of the upper face and surround the circumference of the upper face. In some embodiments, the ramps 117 can be steeper in the clockwise direction than counter-clockwise direction, which limits torque in the opposite direction of the use. In other embodiments, the ramps can be steeper in the counter-clockwise or the ramps can have the same steepness in either direction. In some embodiments, the angle of the ramps can range from approximately 10 to 60 degrees, or 20 to approximately 45 degrees, depending on the application of use and the torque needed for the application.

The thrust washer 114 can be placed between the hub 138 and a bottom face of the lower ratchet 116. In some implementations, the thrust washer 114 can be a long-wearing flat bearing in the shape of a washer that transmits and resolves axial forces in the torque limiting screwdriver 100 thereby keeping the torque-limiting clutch aligned along the shaft 112.

The upper ratchet 120 can include a bearing system 119. In some embodiments, the bearing system 119 comprises a number of ball bearings located on a bottom face thereof. The bearing system 119 can be held in place by the retaining ring 118 and can interface with the ramps 117 of the lower ratchet 116, as will be described more fully below. The top face of the upper ratchet 120 can be in contact with a bottom portion of a compression spring 124 which is retained in position by a spacer 126, compression nut 122 and retaining nut 128.

During manufacture, the thrust washer 114, the lower ratchet 116, the retaining ring 118, the upper ratchet 120, the compression spring 124 and the spacer 126 can be slid onto the shaft. The upper ratchet 120 is then slidably secured to the shaft 112 by placing the upper ratchet retaining screw 132 into the through hole 133. The upper ratchet retaining nut 130 secures the screw in place. The compression nut 122 is then screwed onto the threaded portion of the shaft and tightened until an appropriate amount of force is exerted onto the upper ratchet 120 with respect to the lower ratchet 116. In other words, the torque limiting screwdriver 100 can be calibrated based on the compression spring 124 to limit the amount of force that is applied to an object being driven by the torque-limiting clutch 121. Once the appropriate amount of force is achieved, the retaining nut 128 is tightened against the compression nut 122 so that the compression nut 122 does not loosen during its use.

In use, as the handle 140 is rotated by a user, the handle 140 rotates the shaft so that a screw can be driven into an object. As the screw is driven deeper into the object, a torque is created. As the torque increases it allows the bearing system 119 of the upper ratchet 120 to ride on the ramps 117 of the lower ratchet 16. As the ball bearings 119 ride up the ramps 117, the upper ratchet 120 is pushed toward the compression spring 124. The compression spring 124, in turn, applies a compression force to the upper ratchet 120 that maxes out when the ball bearings 119 reach a top of the ramps 117. This advantageously serves to limit the amount of torque that can be applied to the object. Once the torque limit of the compression spring is achieved, the handle 140 can be rotated by the user but no further torque will be applied to the screw. In other words, if more torque is applied to the handle 140, the bearings 119 will ride the ramps but no torque can be delivered to the shaft tip.

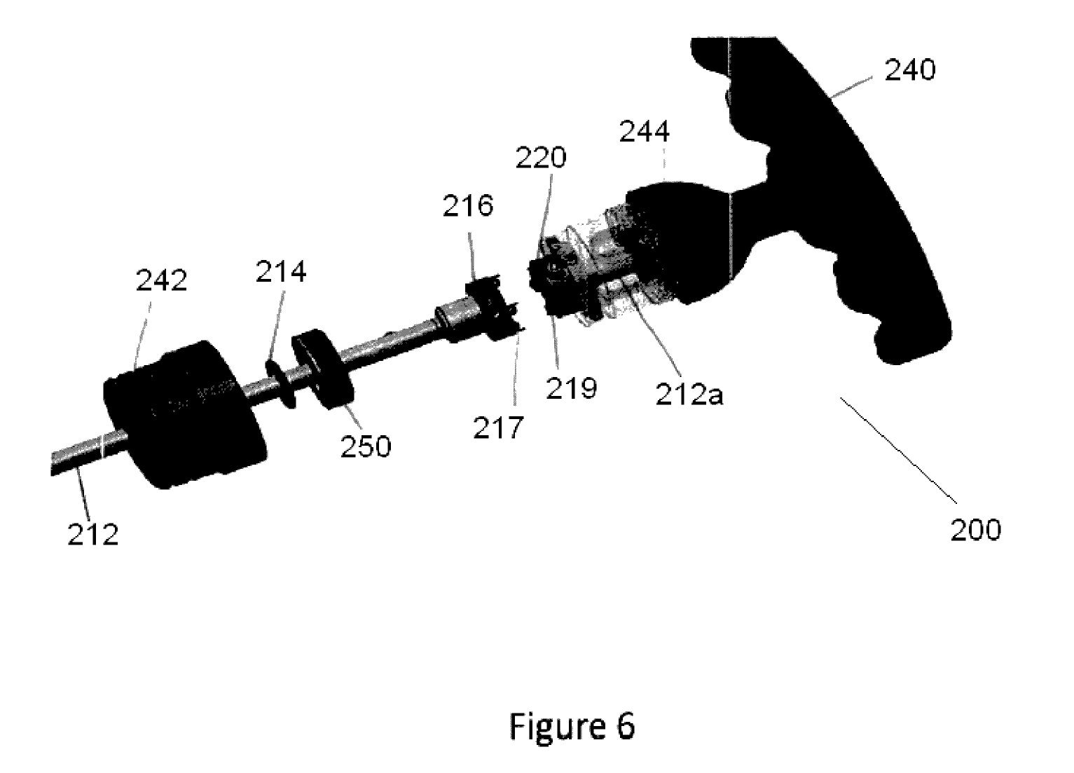

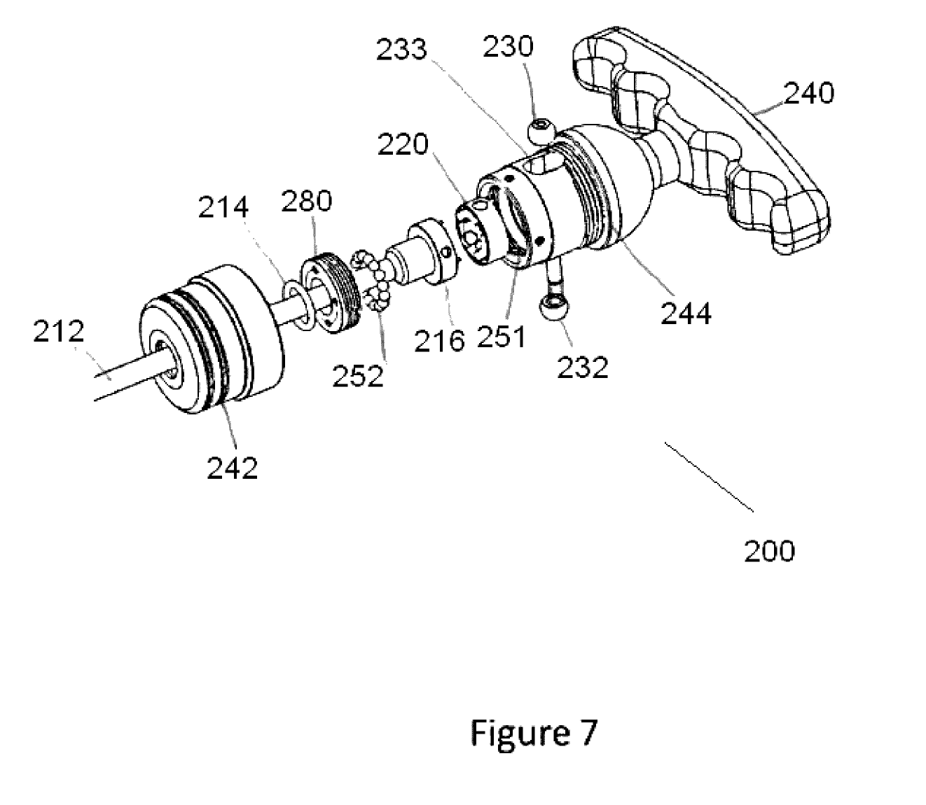

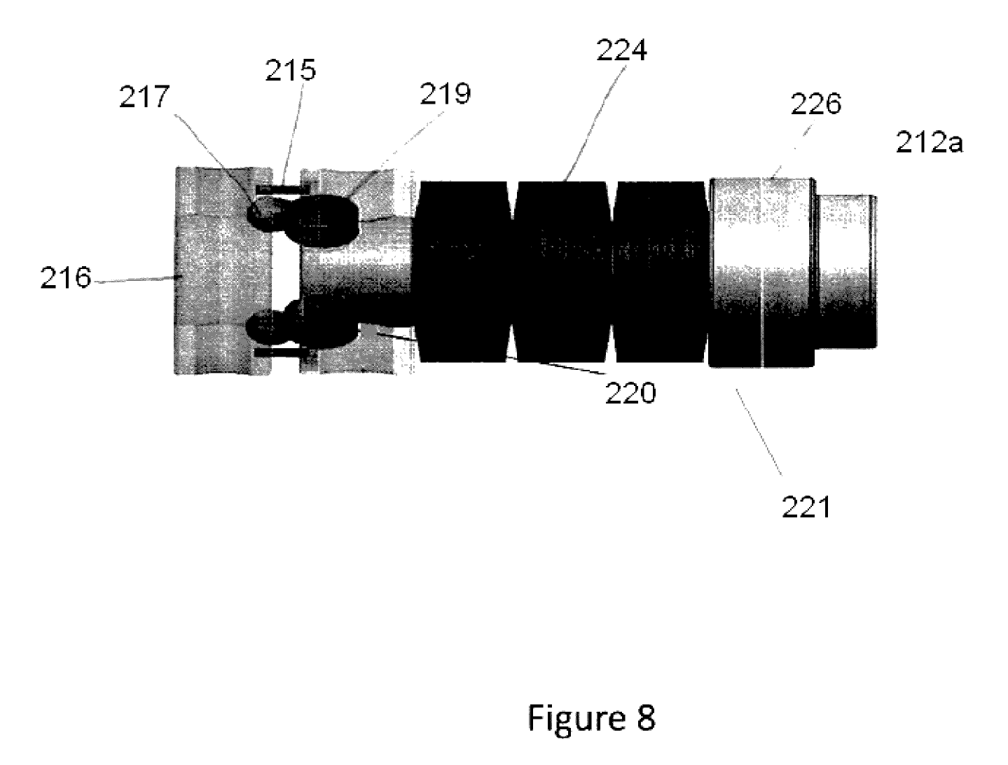

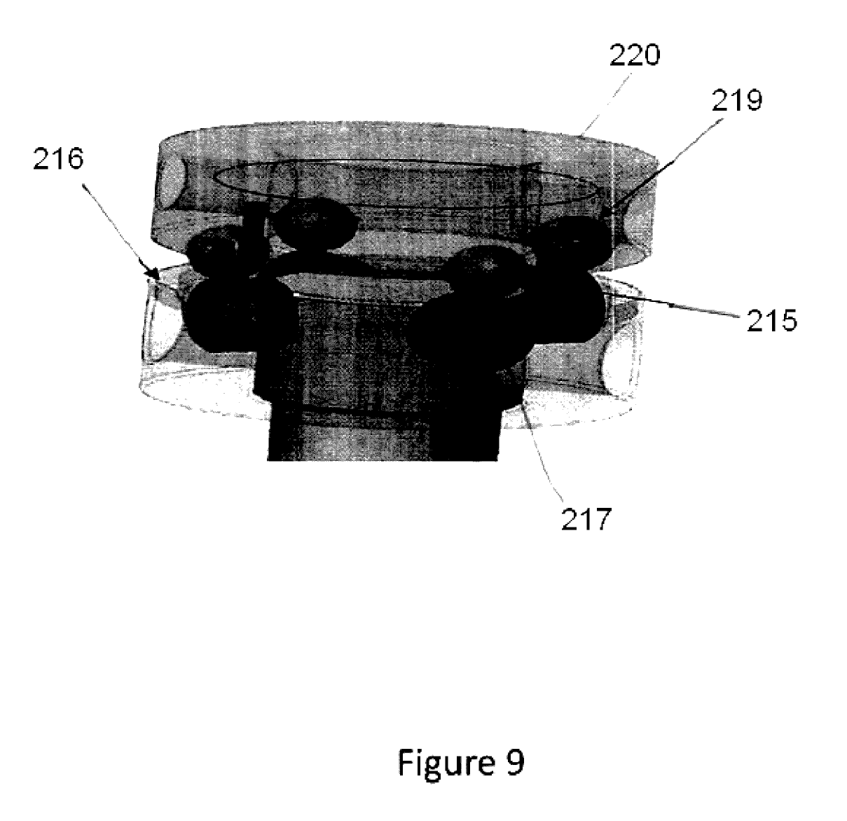

In another implementation of the present disclosure, the driving device can limit torque via an internal ratcheting mechanism utilizing, but not restricted to, opposing bearings producing controlled axial travel, e.g., a clutch mechanism. As shown in FIGS. 6-9, a torque limiting screwdriver 200 is illustrated that is geometrically suited to exert a higher torque load, e.g., via a T-Handle.

The torque limiting T-handle screwdriver 200 has a shaft 212 configured to couple with an such as a head of a surgical fixation element, at one end of the shaft 212. At the other end, the shaft 212 is operably connected to a lower ratchet 216, described more fully below. The torque limiting screwdriver 200 also has a handle 240 that includes an attachment assembly 244 for attaching to handle retainer 242. The attachment assembly 244 can also include a through hole 233 for receiving an upper ratchet retaining screw 232.

In some implementations, the torque limiting screwdriver 200 can also include a torque-limiting clutch 221. The torque-limiting clutch 221 can be used to control an amount of torque being applied to a surgical fixation element via the shaft 212. The torque-limiting clutch 221 can include a thrust washer 214, a torque-limiting clutch retainer 250, a set of bearing 252, a lower ratchet 216, an upper ratchet 220, a compression spring 224, an upper ratchet retaining screw 232 and an upper ratchet retaining nut 230.

In some implementations, the lower ratchet 216 can include discs 217 and backlashes 215 on a top face thereof. That is, the lower ratchet 216 includes a number of discs that are located at the edge of the upper face and surround the circumference of the upper face. Projecting in close proximity to the discs 217 are backlashes 215, described more fully below.

In some implementations, the thrust washer 214 can be placed between the handle retainer 242 and the torque-limiting clutch retainer 250. The thrust washer 214 can be a long-wearing flat bearing in the shape of a washer that transmits and resolves axial forces in the torque limiting screwdriver 200 thereby keeping the torque-limiting clutch aligned along the shaft 212.

In some embodiments, the upper ratchet 220 includes a bearing system 219 such as a number of ball bearings, located on a bottom face therefor. This bearing system 219 can interface with the discs 217 and backlashes 215 of the lower ratchet 216, as will be described more fully below. The top face of the upper ratchet 220 can be in contact with a bottom portion of a compression spring 224.

During manufacture, the lower ratchet 116 can be fixedly secured to a tip of the shaft 212. In some embodiments, the bearing 252, the torque-limiting clutch retainer 250, the thrust washer 214 and the handle retainer 242 are slid onto the shaft. Shaft 212a is fixedly secured to the handle 240 and the compression spring 224 and the upper ratchet 116 are slid on shaft 212a, and the upper ratchet 116 is slidably secured to the attachment assembly 244. The lower ratchet 216 and the upper ratchet 220 are brought in close proximity to each other and the torque-limiting clutch retainer 250 is threadably secured to holder 251. The upper ratchet 120 can then be slidably secured to the attachment assembly 244 by placing the upper ratchet retaining screw 232 into the through hole 233 and the upper ratchet retaining nut 230 secures the screw in place. Also during manufacture, a size of shaft 212a can be chosen so that an appropriate amount of force can be exerted onto the upper ratchet 220 with respect to the lower ratchet 216.

In use, as the handle 240 is rotated by a user, the handle 240 rotates the shaft so that a screw can be driven into an object. As the screw is driven deeper into the object, a torque can be created. As the torque increases it allows the bearing system 219 of the upper ratchet 220 to interact with opposing discs and backlashes 215. The backlashes 215 advantageously prevent the torque-limiting clutch 221 from moving opposite to the direction of its rotation. The placement of the discs and backlashes enables a change in a ratio torque limit with respect to both directions of rotation, clockwise and counter clockwise. As the ball bearings 219 interact with the discs 217, the upper ratchet 220 is pushed toward the compression spring 224. The compression spring 224, in turn, applies a compression force to the upper ratchet that maxes out when the ball bearing are on a top of the discs. This configuration serves to limit the amount of torque that can be applied to the object. Once the torque limit of the compression spring is achieved, the handle 240 can be rotated by the user but no further torque will be applied to the screw. In other words, if more torque is applied to the handle 240, the bearings 219 will ride the discs but no torque can be delivered to the shaft tip.

Various configurations of a torque limiting device are contemplated and are not limited by the embodiments described with reference to the figures. For example, various sizes, shapes and types of torque limiting devices are contemplated, and various materials can be used to construct the various parts, such as handles and clutches described herein. The exemplary embodiments of the present disclosure provide various advantages, such as being able to be limit torque of a driving device. Specifically, torque can be limited via an internal ratcheting mechanism having ramps on one face of the internal ratcheting mechanism that interfaces with bearings contained on another face of the internal ratcheting mechanism. In some implementations, the ramps can be steeper in the direction that the torque will be limited. The ramps can also be manufactured at various angles to change the ratio of a torque limit with respect to both directions of rotation, i.e., clockwise and counter clockwise.

The foregoing merely illustrates the principles of the disclosure. Various modifications and alterations to the described embodiments will be apparent to those skilled in the art in view of the teachings herein. It will thus be appreciated that those skilled in the art will be able to devise numerous apparatuses, arrangements, manufacture and methods which, although not explicitly shown or described herein, embody the principles of the disclosure and are thus within the spirit and scope of the disclosure. The disclosures of all documents and publications cited herein are hereby incorporated herein by reference in their entireties.

What is claimed is:

1. An instrument assembly comprising:
a lower ratchet, the lower ratchet including a plurality of ramps;
an upper ratchet, the upper ratchet including a plurality of bearings; and
a compression spring, the compression spring configured to exert a force on the upper ratchet so that the plurality of bearings interface with the plurality of ramps, wherein the plurality of bearings ride on the plurality of ramps when a torque is created by a rotation of the driving device thereby limiting an amount of torque.

2. The assembly of claim 1 wherein, as the plurality of bearings ride up the plurality of ramps, the upper ratchet is pushed toward the compression spring thereby applying a compression force that maxes out when the plurality of bearings reach a top of the plurality of ramps thereby limiting the amount of torque.

3. The assembly of claim 1 wherein the driving device further comprises a shaft, wherein the torque limiting clutch controls the amount of torque being applied to a surgical fixation element attached to the shaft.

4. The assembly of claim 1 wherein the plurality of ramps are steeper in a clockwise direction than a counter-clockwise direction.

5. The assembly of claim 1 wherein the plurality of ramps are steeper in a counter-clockwise direction than a clockwise direction.

6. The assembly of claim 1 wherein the plurality of ramps have equal steepness in both a counter-clockwise direction and a clockwise direction.

7. The assembly of claim 1 further comprising:
a retaining ring for retaining the plurality of bearings to the upper ratchet.

8. The assembly of claim 3 further comprising:
a thrust washer, the trust washer transmitting and resolving axial forces in the driving device thereby keeping the torque-limiting clutch aligned along the shaft.

9. The assembly of claim 2 further comprising:
a compression nut applying a force to the compression spring so that an appropriate amount of force is exerted onto the upper ratchet with respect to the lower ratchet.

10. The assembly of claim 9 wherein the appropriate amount of force is between approximately 0.5 to approximately 3 pounds of pressure.

11. The assembly of claim 1 wherein the driving device is a screwdriver.

12. The assembly of claim 1 wherein the driving device is a T-Handle screwdriver.

13. The assembly of claim 1 wherein the T-Handle screwdriver is geometrically suited to exert a higher torque load to an object.

14. The assembly of claim 13 wherein the lower ratchet includes backlashes.

15. The assembly of claim 14 wherein the backlashes prevent the torque-limiting clutch from moving opposite its direction of rotation.

16. An instrument assembly comprising:
a shaft, the shaft including an upper section having a proximal end and a distal end and a lower section having a proximal end and a distal end;
a handle, the handle being attached to the proximal end of the upper section;
a torque limiting clutch, the torque limiting clutch including a lower ratchet having a plurality of discs and backlashes and an upper ratchet having bearing assembly, the lower ratchet being attached to the proximal end of the lower section and the upper ratchet being attached to the distal end of the upper section; and
a compression spring, the compression spring being placed on the upper section of the shaft between the upper ratchet and the handle, the compression spring exerting a force on the upper ratchet so that the bearing assembly interfaces with the plurality of discs and backlashes.

17. The assembly of claim 16, wherein the bearing assembly rides on the plurality of discs when a torque is created by a rotation of the driving device.

18. The assembly of claim 16, wherein, as the bearing assembly rides up the plurality of discs, the upper ratchet is pushed toward the compression spring thereby applying a compression force that maxes out when the bearing assembly reaches a top of the plurality of discs thereby limiting an amount of torque.

19. The assembly of claim 16, wherein the driving device is a T-Handle screwdriver geometrically suited to exert a higher torque load to an object.

20. The assembly of claim 16, wherein the backlashes prevent the torque limiting clutch from moving opposite its direction of rotation.
